(19) United States
(12) Patent Application Publication
(10) Pub. No.: US 2004/0219519 A1
(43) Pub. Date: Nov. 4, 2004

(54) Method for bacteriophage delivery and amplification

(76) Inventors: Billy M. Hargis, Fayetteville, AR (US); Stacy E. Higgins, Fayetteville, AR (US); Lisa Bielke, Fayetteville, AR (US); Guillermo I. Tellez, Fayetteville, AR (US); Lisa A. Newberry, Benton, AR (US); Gerardo Nava, Fayetteville, AR (US); Ann M. Donoghue, Springdale, AR (US); Daniel J. Donoghue, Springdale, AR (US)

Correspondence Address: Ray F. Cox, Jr., WRIGHT, LINDSEY & JENNINGS LLP, Suite 2300, 200 West Capitol Avenue, Little Rock, AR, 72201-3699, US

(21) Appl. No.: 10/818,273

(22) Filed: Apr. 5, 2004

Related U.S. Application Data
(60) Provisional application No. 60/461,042, filed on Apr. 7, 2003.

(51) Int. Cl. C12Q 1/70; A61K 45/00

(52) U.S. Cl. 435/5; 424/93.6

(57) ABSTRACT

We recently isolated bacteriophages from wastewater that lyse a primary poultry isolate of Salmonella enteritidis (SE). We evaluated the host range bacteriophage isolates and found that they have the ability to also lyse up to 7 other Salmonella isolates, as well as E. coli, and Klebsiella sp. when plated using soft agar overlay technique. Further, we found that addition of bacteriophage to carcass rinse samples significantly reduced the amount of recoverable Salmonella in both laboratory and field trial settings. The invention also comprises selecting appropriate bacteriophages that target a specific pathogen, and identifying beneficial microflora that would serve as an alternative host. We were able to identify several such alternative hosts from the beneficial microflora selected in our competitive exclusion project. We have also shown that by allowing a brief incubation of the selected bacteriophages and the alternative bacterial hosts, the bacteriophages are protected from destruction in the environment of the upper gastrointestinal tract. This invention provides a very inexpensive and highly effective alternative to traditional chemotherapy with regard to controlling bacterial infections of the gastrointestinal tract of poultry, other animal species, aquaculture applications, and humans.

### CROSS-REFERENCE TO RELATED APPLICATIONS

This application claims the benefits of U.S. Provisional Application No. 60/461,042 filed Apr. 7, 2003, which is incorporated herein by reference.

### STATEMENT REGARDING FEDERALLY SPONSORED RESEARCH OR DEVELOPMENT

This invention was made with Government support under the terms of Grant No. 2001-34211-10288 awarded by the United States Department of Agriculture, Cooperative State Research Education and Extension Service. The Government has certain rights in the invention.

### BACKGROUND OF THE INVENTION

Foodborne illness affects more than 76 million Americans each year, with salmonellosis contributing to 36% of this incidence, with an estimated cost of 1.4 billion dollars in lost human productivity, medical expenses and increased animal production costs in the United States alone (FoodNet, 2002; Madie, 1992). Specific bacteriophage therapy has been demonstrated in a number of research studies to be potentially efficacious as an alternative to the use of antimicrobial drugs for the control of enteric disease in animals and man (Slopek et al., 1987; Smith and Huggins, 1982, 1983; Park et al., 2000, Huff et al., 2001).

Bacteriophages are members of a specific Kingdom of viruses that only infect bacteria, with no potential to infect animals or plants (Carlton, 1999). In both published literature and textbooks that address bacteriophage biology, the overwhelming indication and belief is that these viruses are extremely host specific and will infect and replicate in a limited number of closely related bacteria. There have been numerous attempts, with some success, of using-bacteriophages to treat bacterial infections (Slopek et al., 1987; Smith and Huggins, 1982, 1983; Park et al., 2000, Huff et al., 2001). However, commercial use of bacteriophages for treatment of enteric infections has not gained widespread acceptance, partially because of difficulties delivering large numbers of bacteriophages to the sites of enteric infection. For example, bacteriophages are lost as they travel through the upper gastrointestinal tract, which has uniformly resulted in the need for administration of large numbers of bacteriophages for the treatment of enteric infection (see above references).

We have demonstrated that a surprising number of wild-type bacteriophage isolates can be readily propagated in some relatively non-related bacteria. The invention comprises selecting appropriate bacteriophages that target a specific pathogen, and identifying non-pathogenic or beneficial microflora that would serve as an alternative host.

### BRIEF SUMMARY OF THE INVENTION

We recently isolated bacteriophages from wastewater that lyse a primary poultry isolate of Salmonella enteritidis (SE). We evaluated the host range bacteriophage isolates and found that they have the ability to also lyse up to 7 other Salmonella isolates, as well as 3 species of E. coli and 1 Klebsiella sp. when plated using soft agar overlay technique. Further, we found that addition of bacteriophage to carcass rinse samples significantly reduced the amount of recoverable Salmonella in both laboratory and field trial settings.

The invention also comprises selecting appropriate bacteriophages that target a specific pathogen, and identifying beneficial microflora that would serve as an alternative host. We were able to identify several such alternative hosts from the beneficial microflora selected in our competitive exclusion project. We have also shown that by allowing a brief incubation of the selected bacteriophages and the alternative bacterial hosts, the bacteriophages are protected from destruction in the environment of the upper gastrointestinal tract. This invention provides a very inexpensive and highly effective alternative to traditional chemotherapy with regard to controlling bacterial infections of the gastrointestinal tract of poultry, other animal species, aquaculture applications, and humans.

### BRIEF DESCRIPTION OF THE SEVERAL VIEWS OF THE DRAWINGS

These and other features, objects and advantages of the present invention will become better understood from a consideration of the following detailed description, appended claims and accompanying drawings where:

FIG. 1 is a graph showing amplification of Salmonella enteritidis-selected bacteriophages in alternative host bacteria. Bacteriophages isolated from waste-water treatment samples were selected based on ability to form plaques in Salmonella enteritidis soft-overlay plates. The combined bacteriophages were then amplified in an alternative host bacteria (E. coli-3 isolates, Klebsiella sp.-1 isolate). The plaque forming units (PFU) were determined in Salmonella enteritidis using soft agar overlay plates.

FIG. 2 is a graph showing amplification of wide-host-range bacteriophages in alternative hosts. Bacteriophages from Salmonella enteritidis soft agar overlay plates after enrichment in the alternative host bacteria were isolated and re-amplified in the alternative host. The bacteriophages were then plated using the soft agar overlay method and the respective alternative host bacteria.

FIG. 3 is a graph showing recovery of Salmonella enteritidis (SE) from broiler carcasses inoculated with 31 cfu of SE, and sprayed with 5.5 mL of 0 or 10¹⁰ pfu/mL of a single bacteriophage isolate. Bacteriophages may be amplified in alternative host bacteria as described in FIGS. 1 and 2, eliminating the risk of accidental introduction of a pathogenic bacterium to a food product. ^{a-b} Different superscripts indicate significant (P<0.05) differences in recovery incidence.

FIG. 4 is a graph showing recovery of Salmonella enteritidis (SE) from broiler carcasses inoculated with 20 cfu of SE, and sprayed with 5.5 mL of 0 or 0.53×10⁴, 106, 108, or 1010 pfu/mL of a single bacteriophage isolate. Bacteriophages may be amplified in alternative host bacteria as described in FIGS. 1 and 2, eliminating the risk of accidental introduction of a pathogenic bacterium to a food product. ^{a-b} Different superscripts indicate significant (P<0.05) differences in recovery incidence.

FIG. 5 is a graph showing recovery of Salmonella from commercial turkey carcasses rinsed with saline (control), saline containing 72 bacteriophage isolates amplified either in the original Salmonella enteritidis host (1.6×10⁸ pfu/mL), in the Salmonella field isolate (S9) obtained from the flock antemortem (1.8×10⁷ pfu/mL) or a combination of both (8.9×10⁷ pfu/mL). Bacteriophages may be amplified in alternative host bacteria as described in FIGS. 1 and 2, eliminating the risk of accidental introduction of a pathogenic bacterium to a food product. ^{a-b} Different superscripts indicate significant differences (P<0.05) in incidence between groups.

FIG. 6 is a graph showing recovery of Salmonella from commercial turkey carcasses rinsed with saline (control), saline containing bacteriophage isolates amplified either in the original Salmonella enteritidis host (8.0×10⁷ pfu/mL), the Salmonella field isolate (S14) obtained antemortem (9.0×10⁶ pfu/mL), or a combination of both (4.5×10⁷ pfu/mL). Bacteriophages may be amplified in alternative host bacteria as described in FIGS. 1 and 2, eliminating the risk of accidental introduction of a pathogenic bacterium to a food product. ^{a-b} Different superscripts indicate significant differences (P<0.05) in incidence between groups.

### DETAILED DESCRIPTION OF THE INVENTION

Amplification of bacteriophages for use in treatment of animal or human infections, or treatment of products intended for use in food or feed, has traditionally carried some risk of introducing the target pathogenic bacterium accidentally if separation of bacteriophages from host bacteria is not completely assured. The ability to amplify bacteriophages in alternative non-pathogenic hosts greatly reduces this potential threat, and the costs associated with redundant steps to assure exclusion of the amplification host from the bacteriophage product. By amplification of prophylactic or therapeutic bacteriophages in non-pathogenic hosts during manufacture, risk of introduction of pathogenic bacteria is eliminated. Similarly, alternative hosts which produce little or no endotoxins or exotoxins can be selected for amplification of therapeutic bacteriphage under commercial conditions, thereby reducing or eliminating the need for purification (removal of toxins) for use of such preparations on food, feed, animals or humans.

Indeed one major obstacle has proven to be related to tremendous reductions of viable bacteriophages as they pass through the relatively low pH conditions of the upper gastrointestinal tract. Initial success in using bacteriophage therapy for treating enteric salmonellosis has only been achieved with simultaneous administration of very large numbers of bacteriophages with a buffering solution.

As described below, we have demonstrated, 1) new approaches for selection and application of appropriate bacteriophages for treating enteric bacterial infections, and 2) novel approaches for selection and application of alternative hosts for the bacteriophage. This combined approach provides a powerful and cost-effective tool to prevent or treat infectious bacterial diseases, including enteric diseases.

The invention comprises selecting appropriate bacteriophages that target a specific pathogen. The invention further comprises identifying beneficial microflora that would serve as an alternative host for the bacteriophage. The invention also pertains to the development of alternatives to antimicrobial chemicals for control of enteric bacterial infections of poultry, other domestic animals, and humans. The invention also pertains to using animals as a pass-though mechanism to identify and select beneficial microflora.

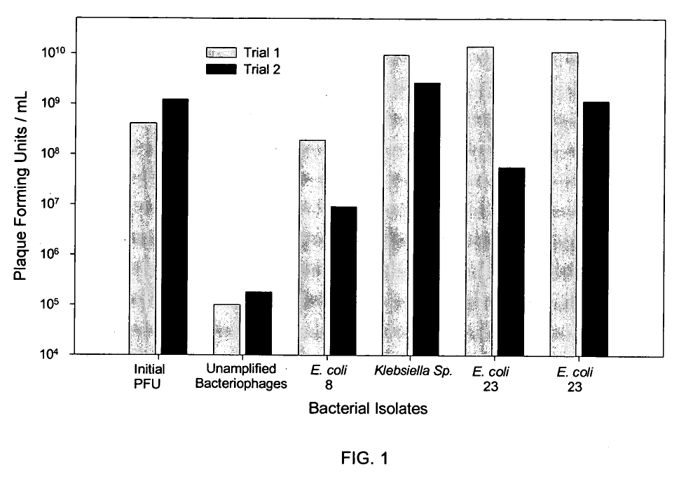

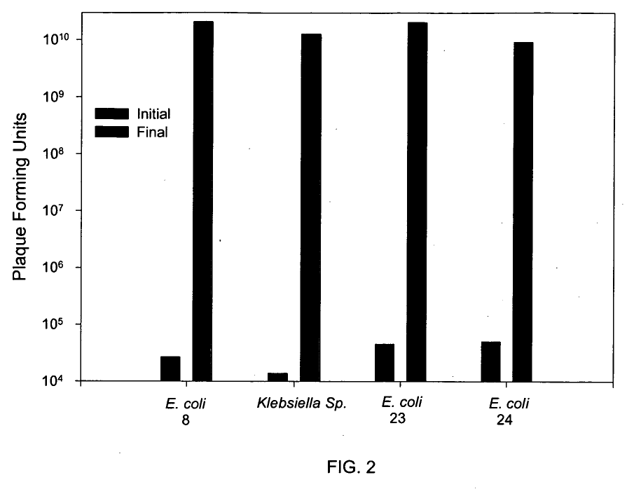

We have developed a method to select bacteriophages and beneficial microflora that would serve as an alternative host for the bacteriophage, for effective therapeutic use in poults. Using poults as a biological filter, we administered bacteriophages orally and then recovered phages from the ileum and ceca. We selected phages by three serial passages in three poults each passage. Early cultures provided marked protection against Salmonella infection. Studies indicate that our now-established and partially-characterized bacteriophages (72 isolates) are capable of reducing Salmonella recovery, and potentially other enteric bacteria pathogens, from the intestine (Tables 1 and 2) (FIGS. 1 and 2).

TABLE 1
Recovery of Salmonella enteritidis (SE) from cecal contents of poults
challenged with 1 × 10⁴ cfu of SE 48 hours after placement,
and treated with 2.5 × 10⁹ pfu
of selected bacteriophages 48 h post-challenge.

| | Average colony forming units (cfu) of SE per gram of cecal contents | | | |
|---|---|---|---|---|
| | recovered | | | |
| Treatment | 6 h¹ | 12 h | 24 h | 48 h |
| Control | 21,329 ± 12,694 | 11,294 ± 5,699 | 29,812 ± 21,506 | 5,189 ± 2,739 |
| Bacteriophage | 20,840 ± 6,758 | 44,162 ± 27,811 | 30,437 ± 18,118 | 55,288 ± 13,557 |
| Mg(OH)₂ and | 41,962 ± 4,402 | 1,437 ± 765 | 4,064 ± 2,299 | 227,527 ± 131,210 |
| bacteriophage | | | | |

¹Time of culture is number of hours indicated after treatment.
²0.25 mL of 5 mM Mg(OH)₂ solution was administered 30 min before bacteriophage administration. Bacteriophage suspension also contained 1 mM Mg(OH)₂.

TABLE 2
Recovery of Salmonella enteritidis (SE) from cecal contents of
poults challenged with 1.6 × 10⁴ cfu of SE 48 hours after
placement, and treated with 7.5 × 10⁹ pfu of selected
bacteriophages 48 h post-challenge.

| | Average colony forming |
|---|---|
| | units of SE per gram |
| Treatment | cecal contents 24 h post-treatment |
| Control | 79,728 ± 25,893 |
| Mg(OH)₂ and bacteriophage | 11,224 ± 9,110 |

The invention also comprises utilizing the method of providing the beneficial bacterial cultures (alternative hosts) continuously in the drinking water after bacteriophage/alternative host administration (Tables 3 and 4). By this method, very high levels of these appropriate bacteriophages in the gastrointestinal tract are maintained. Experiments have proven remarkably effective at prevention of young poultry from a model Salmonella enteritidis enteric infection (FIGS. 1 and 2). In these studies, large numbers of bacteriophages were isolated from the ceca of birds treated with a combination of the alternative host bacterial culture and bacteriophages.

TABLE 3
Effect of Combination Treatment of Bacteriophage (Ø)
and a Beneficial Bacterial Culture Serving as an
Alternative Bacteriophage Host (Mean cfu of
SE/g cecal contents in log₁₀ units)

| Group | Treatment | 24 h | 48 h | 72 h |
|---|---|---|---|---|
| 1 | Control | 6.02 ± 5.40 | 5.53 ± 5.15 | 4.78 ± 4.30 |
| | Challenged with | | | |
| | SE day 1 | | | |
| 2 | Prophylactic | 0 | 0 | 0 |
| | Ø + FN gavage | | | |
| | FN in H₂O | | | |
| 3 | Prophylactic | 0 | 0 | 0 |
| | Ø + FN gavage | | | |
| | Ø + FN in water | | | |

TABLE 4
Effect of Combination Treatment with Bacteriophage (Ø) and
a Beneficial Bacterial Culture Serving as an
Alternative Bacteriophage Host

| | | Mean cfu of SE/g | Number of |
|---|---|---|---|
| | | cecal contents in | positive |
| | | log₁₀ units 24 h | samples/ |
| Group | Treatment | post treatment | total samples |
| 1 | Control | 5.47 ± 5.05 | 18/20 |
| | Challenged with SE day 1 | | |
| 2 | Prophylactic | 5.53 ± 5.20 | 10/10 |
| | FN gavage | | |
| | FN in H₂O | | |
| 3 | Prophylactic | 3.76 ± 3.41 | 5/10* |
| | Ø + FN gavage | | |
| | FN in water | | |

*Significant (P < 0.05) difference in recovery incidence

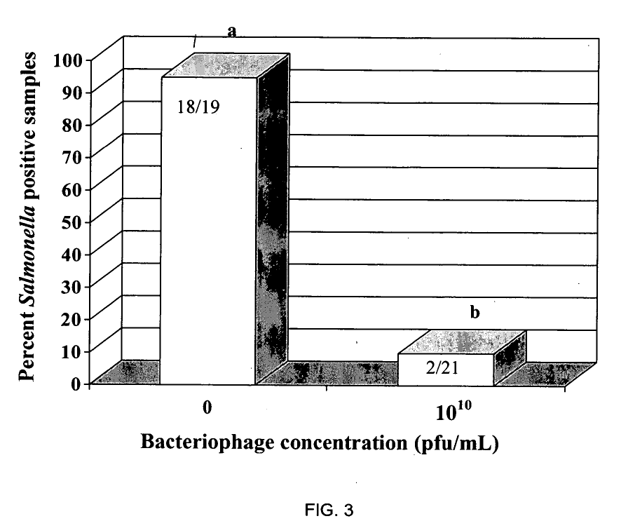

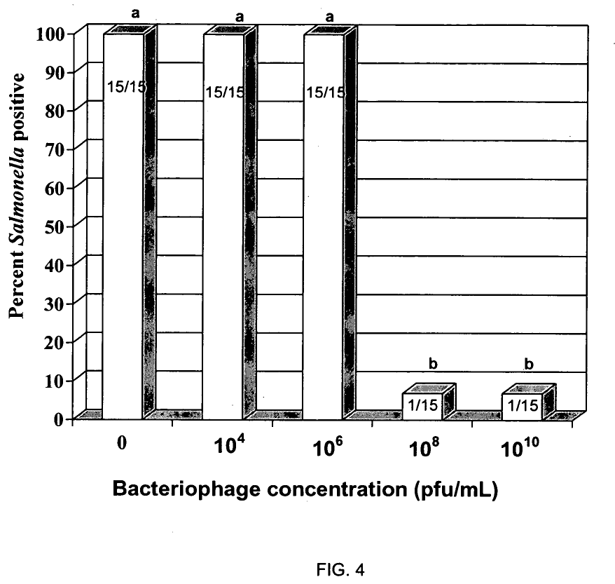

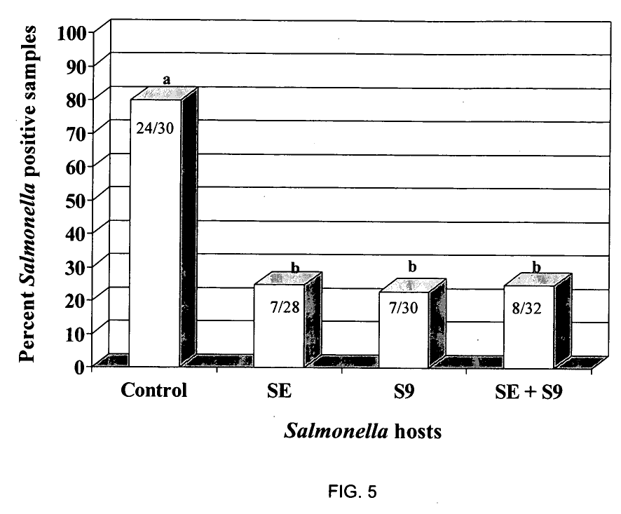

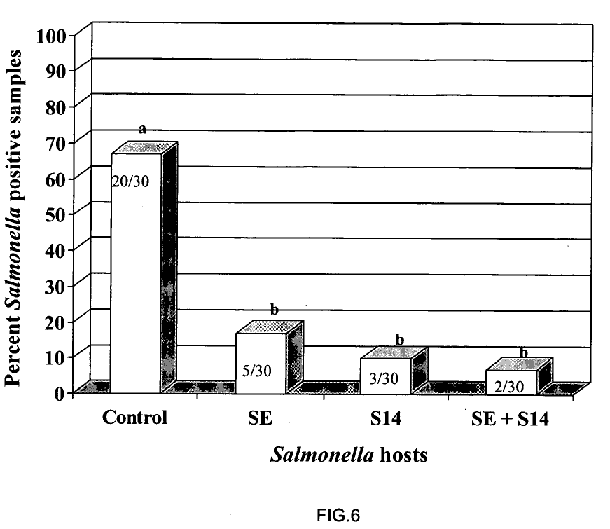

Commercially-processed carcasses were rinsed and the resulting medium was pooled and divided. Each rinse sample was simultaneously inoculated with SE and a single-wild-type phage as described below. Broiler carcasses were intentionally inoculated with SE (31 or 20 cfu per carcass) and sprayed with 5.5 mL of 10¹⁰ pfu/mL phage or 0.53×10⁴, 10⁶, 10⁸, or 10¹⁰ pfu/mL phage, followed by traditional enrichment culture for Salmonella (FIGS. 3 and 4). Recovery of Salmonella from commercial turkey carcasses rinsed with saline, saline containing 72 bacteriophage isolates amplified either in the original Salmonella enteritidis host, in the Salmonella field isolate (S9) obtained from the flock antemortem or a combination of both is shown in FIG. 5. Commercially processed turkeys were rinsed with saline, saline containing bacteriophage isolates amplified either in the original Salmonella enteritidis host, the Salmonella field isolate (S14) obtained antemortem, or a combination of both is shown in FIG. 6.

### EXAMPLES

Bacteriophage Isolation. Approximately 72 bacteriophage isolates were obtained by traditional soft-overlay plating and identification of lytic plaques. Resulting plaques were passed on agar plates three times to ensure homogeneity of each isolate.

Host Specificity. Bacteriophage isolates were amplified to a high titer using a ratio of 1:3:5 (bacteriophage: plateau phase SE in tryptic soy broth (TSB): fresh TSB), and incubated at 37 C for 1.5 h. After amplification, bacteriophages (PHL 4 and PHL 5) were individually plated at 10× dilutions with 11 different Salmonella isolates (1×10⁷ cfu/ml) in soft agar overlay plates. The plates were allowed to solidify and incubated overnight at 37 C. The number of plaques formed on each host organism were then quantified.

Therapeutic Treatment In Vivo. Bacteriophages were selected for pH tolerance and ability to survive the gastrointestinal tract of chicks by passage through three chicks each time for three serial passages. Briefly, a mixture of wild-type isolates 1-71 was administered to SE infected poults by oral gavage. When a feed passage marker was observed in the feces, the poults were killed, and the ileum, cecal tonsils, and cecae were removed and enriched for 30 mm with TSB and SE. Phages isolated following the first passage were administered to the second group of poults. Bacteriophages were similarly recovered and again passaged two times. The resulting mixture of bacteriophages was used to treat poults in two replicate experiments.

What is claimed is:

1. A method of selecting and amplifying bacteriophages targeting a specific enteric pathogen in an animal species, comprising the steps of:
(a) isolating a group of wild bacteriophages from environmental or waste water treatment samples;
(b) selecting enteric pathogen-specific bacteriophages from said group of wild bacteriophages based on the ability of said wild bacteriophages to form lytic plaques in soft agar overlay plates inoculated with said enteric pathogen;
(c) selecting a group of non-pathogenic bacteria as potential alternative hosts to said enteric pathogen-specific bacteriophages;
(d) amplifying a combination of said enteric pathogen-specific bacteriophages in said non-pathogenic bacteria; and
(e) passing said combination of enteric pathogen-specific bacteriophages in said non-pathogenic bacteria through the gastrointestinal tract of at least one individual of said animal species.

2. The method of claim 1 where the animal species is a species of domestic animal.

3. The method of claim 1 where the animal species is a species of poultry.

4. The method claim 1 where the animal species is a species of fish.

5. The method of claim 1 where the animal species is a human.

6. The method of claim 1 where the enteric pathogen is a species of Salmonella.

7. The combination of enteric-specific bacteriophages in non-pathogenic bacteria of claim 1.

8. The combination of enteric-specific bacteriophages of claim 1.

9. The combination of non-pathogenic bacteria of claim 1.

10. A method of treating a living individual of an animal species to reduce incidence of said enteric pathogen by oral administration to said individual with the combination of non-pathogenic bacteria of claim 9 following inoculation of said individual with the combination of enteric pathogen-specific bacteriophages in non-pathogenic bacteria of claim 7.

10. The method of claim 10 where the animal species is a species of domestic animal.

11. The method of claim 10 where the animal species is a species of poultry.

12. The method claim 10 where the animal species is a species of fish.

13. The method of claim 10 where the animal species is a human.

14. The method of claim 10 where the enteric pathogen is a species of Salmonella.

15. A method of treating a food carcass of an individual of an animal species or food containing meat of said carcass to reduce incidence of said enteric pathogen by spraying the combination of enteric pathogen-specific bacteriophages of claim 8 onto said carcass.

16. The method of claim 15 where the animal species is a species of domestic animal.

17. The method of claim 15 where the animal species is a species of poultry.

18. The method claim 15 where the animal species is a species of fish.

19. The method of claim 15 where the enteric pathogen is a species of Salmonella.
